(19) United States
(12) Patent Application Publication
(10) Pub. No.: US 2004/0147719 A1
(43) Pub. Date: Jul. 29, 2004

(54) Type III bacterial strains for use in medicine

(76) Inventor: Guy Cornelis, Kraainem (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/473,328

(22) PCT Filed: Mar. 26, 2002
(86) PCT No.: PCT/EP02/03372

(30) Foreign Application Priority Data
Mar. 26, 2001 (EP) 01870064.1

(51) Int. Cl. C12N 1/20; C07K 1/00; C07K 14/00; C07K 17/00

(52) U.S. Cl. 530/350

(57) ABSTRACT

The present invention relates to a safe non-virulent Yersinia enterocolitica mutant strain for delivering heterologous proteins in target cells carrying mutations in at least one of the effector genes yopH, yopO, yopP, yopE, yopM, yopT genes and at least one additional mutation in the invasin genes chosen from yadA and/or inv. The present invention also relates to a safe non-virulent Yersinia enterocolitica mutant strain for delivering heterologous proteins in target cells according to claim 1 carrying mutations in all effector genes yopH, yopO, yopP, yopE, yopM, yopT genes and at least one additional mutation in the invasin genes chosen from yadA and/or inv. The present invention also relates to an expression vector for delivering a heterologous protein into a target cell using a Yersinia enterocolitica mutant strain according to any of the claims 1 to 4, which comprises in the 5′ to 3′ direction: (a) a promoter of a Yersinia virulon gene, (b) a first DNA sequence encoding a delivery signal from a Yersinia effector protein, operably linked to said promoter; and, (c) a second DNA sequence coding for said heterologous protein, fused in frame to the 3′ end of said first DNA sequence. The present invention further relates to methods and compositions comprising (the use of) the afore-mentioned mutant strains and expression vectors.

### TECHNICAL FIELD

The present invention relates to optimised bacteria in particular to improved type III strains for use in medicine. The present invention further relates to a method for classifying type-III bacteria.

### BACKGROUND ART

For a rather long period of time, it was assumed that Gram-negative bacteria do not secrete proteins into their environment. It has been shown later on that Gram-negative bacteria can transfer proteins across their sophisticated outer membrane using specialized systems. One of these systems, type III, is a sophisticated apparatus that couples secretion with pathogenesis.

The type III system of Yersinia (Yop) is considered to be the archetype of a whole family of systems encountered in several gram-negative animal and plant pathogens. Other pathogenic bacteria sharing very similar type III systems are: Pseudomonas aeroginosa, Bordetella pertussis (whooping cough), Salmonella, Shigella, enteropathogenic E.coli, Burholderia cepacia and Chlamydia.

The three Yersinia species that are pathogenic to man, Y. pestis, Y. pseudotuberculosis and Y. enterocolitica, all share in common the ability to deliver bacterial effector proteins called “Yops” inside eukaryotic cells. Y. enterocolitica is a common contaminant of pork meat and an agent of mild diarrhea and abdominal pain and is frequently used to study the type III machinery. The Yop effectors allow the bacteria to survive and proliferate extracellularly in the gut-associated lymphoid system of the host. The Yop effector proteins and their machinery for export from the bacteria and transport into the eukaryotic cell are all encoded by a 70 kb virulence plasmid—which is called pYV in Y. enterocolitica. This whole system, along with its regulatory circuit, is called the Yop virulon (reviewed in Cornelis 2000).

The Yop virulon is a sophisticated pathogenicity system used by bacteria from the genus Yersinia (Y. enterocolitica, Y. pseudotuberculosis and Y. pestis) to defeat the immune system of their host. Yersinia are not detected inside the inflammatory or parenchymal cells of the infected animals and have the capacity to enter certain cultured epithelial cells, a process generally referred to as an invasion. This system allows extracellular bacteria adhering at the surface of eukaryotic cells to inject bacterial effector proteins into the cytosol of these cells in order to disarm them or disrupt their communications.

The Yersinia virulon is composed of the following elements: 1/a contact or type III secretion system (Yst) which is devoted to the secretion of Yop proteins out of the bacterial cells; 2/a set of “translocators” for translocating the effector proteins into the eukaryotic host (YopB, YopD, LcrV and LcrG) 3/a control element and recognition system (YopN and LcrG); and (iv) a set of “effector proteins” including YopE, YopH, YopO/YpkA, YopM, YopT and YopP/YopJ, which are injected (or translocated) into the eukaryotic host cells and disrupt the functions of such host cells.

Transcription of these genes is controlled both by temperature and by contact with an eukaryotic cell. When Yersinia is placed at 37° C. in a rich environment, the Ysc secretion apparatus is installed and a stock of Yop proteins is synthesized. As long as there is no contact with an eukaryotic cell, a stop-valve (YopN) prevents secretion. Upon contact with a target, the Ysc apparatus opens up and Yops are delivered. During their intrabacterial storage, some Yops are capped with a specific Syc chaperone carrying an anti-degradation role and regulating the hierarchy for delivering the effectors.

Six Yop effectors are known in Yersinia—YopE, YopH, YopM, YopT, YopO/YpkA and YopP/YopJ. The effector proteins disrupt the function of host cells in a number of ways. Out of the six proteins delivered by Yersinia, three (YopH, YopE, YopT) exert a negative role on cytoskeleton dynamics and contribute to the strong resistance of Yersinia to phagocytosis by macrophages (Cornelis and Van Gijsegem, 2000). The 23 kd YopE is a cytotoxin that disrupts the actin-microfilament structure of cultured Hela cells. The disruption of these actin filaments by YopE leads to inhibition of phagocytosis by polymorphonuclear leukocytes (PMNs) and macrophages. The 51 kd YopH is a protein tyrosine phosphatase (PTPase) related to eukaryotic PTPases, which acts on tyrosinephosphorylated proteins of infected macrophages. Dephosphorylation of these proteins leads to disruption of the signal transduction pathways involved in cellular uptake of bacteria, possibly by specifically inhibiting self-induced calcium-signaling. Presumably as a result of this action, YopH inhibits bacterial uptake and oxidative burst by cultured macrophages. In addition, YopH suppresses T- and B-lymphocyte activation via their antigen receptors. T-cells are impaired in their ability to produce cytokines and B-cells are unable to upregulate surface expression of the co-stimulatory molecule B7.2 in response to antigen activation. Cytotoxin YopT inactivates a GTPase that regulates the formation of stress fibers. YopO (or YpkA) is an 81 kd serine/threonine kinase, which is targeted to the inner surface of the plasma membrane of the eukaryotic cell and might function to interfere with the signal transduction pathway of the eukaryotic cell. YopM is an acidic 41 kd protein having 12 leucine-rich repeats. The function of YopM is not well understood as well as the function of similar proteins from other type III containing bacteria. YopP (also called YopJ in Y. pestis and Y. pseudotuberculosis) induces apoptosis of macrophages. YopP also blocks the release of TNFα by macrophages and IL-8 by epithelial cell, which leads to a significant reduction in inflammation.

The Yop effectors require the Ysc proteins in order to cross the two bacterial membranes. The latter constitute the Ysc apparatus, which is supposed to form some kind of a needle. Four “translocators” are essential for the translocation of the effector Yops across the eukaryotic membrane: YopB, YopD, LcrV and LcrG. Two of them, YopB and YopO form a pore in the eukaryotic cell membrane.

In order for Y.enterocolitica to translocate Yops into eukaryotic cells the bacteria must adhere to the surface of eukaryotic cells. The attachment induces opening of the Ysc secretion channel and increased production of the Yops. The two major determinants of adhesion in Y. enterocolitica are Invasin and YadA. Invasin is a chromosomally encoded protein which binds β1 integrins on the surface of eukaryotic cells, while YadA is encoded by the virulence plasmid itself and binds a variety of extracellular proteins including fibronectin and collagen. There may be a receptor on the eukaryotic cell surface that interacts with a ligand on the bacterial surface and leads to a signal for Yop injection. In Y. enterocolitica YopN, TyeA and/or LcrG have the potential to be the bacterial ligand, as bacteria carrying mutations in any one of these genes have a deregulated Yop secretion phenotype.

Translocation of the Yop effectors into eukaryotic cells could be demonstrated using hybrid proteins fusing truncated Yop effector proteins of different length with certain reporter enzymes such as the calmodulin-activated adenylate cyclase domain (Cya) of the Bordetelia pertussis cyclolysin. This illustrated that Yops are translocated into eukaryotic cells but also that Yersinia can deliver foreign proteins into eukaryotic cells (or Cya). By applying this approach, it was suggested in U.S. Pat. No. 5,965,381 that the N-terminal domain of YopE, YopM, YopH, YopO/YpkA, and YopP/YopJ of Y. enterocolitica could be used as “delivery signal” to deliver heterologous proteins in eukaryotic cells. In this patent the use of such expression vector in combination with a mutant Yersinia strain to deliver a desired protein into eukaryotic cells was demonstrated. This mutant Yersinia was defined to be deficient in the production of functional effector proteins, but still endowed with a functional secretion and translocation system. This approach was found to be useful not only for studying the function of a given protein, but also for designing therapeutic approaches. The inventors pointed out that these mutant Yersinia had a diminished toxicity, i.e., toxicity which does not completely disable or kill the target cell. Nevertheless, when using recombinant strains for medical purposes it is of great importance that the strains used are safe to use. Its is also of prime importance that, especially when starting from a virulent strain, all virulent factors are deleted.

In addition, it has been recently shown that the translocation of the Yop effector proteins using Yersinia enterocolitica is not selective (Boyd at al. 2000). This would suggest that, although this system seemed to be a potential vector for delivering heterologous proteins into eukaryotic cells, the delivery is more or less random. Unfortunately, such a non-selective delivery may elicit unwanted side effects of the medicament comprising this mutant strain; this is not be permitted in medicine.

### OBJECTS OF THE INVENTION

The present invention is therefore directed towards the development of an improved Yersinia vector strain, whereby “improved” means that the strain is much safer to use in medicine. With “safe” is meant that virulence of the strain is reduced to a minimun, creating a non-harming delivering vector; in addition, “safer” also means that the corresponding strain does not include any factor which is not allowed by the FDA. With “vector strain” is meant a cell which is used to deliver genetic information (DNA or protein) in another cell. The advantage of a bacterial vector strain versus a viral vector is that antibiotics may still be used to eliminate the vector strain in a subject when it is not needed anymore; this is not possible when using viral vectors.

A particular object of the present invention is directed towards the development of a well considered Y.enterocolitica which is safe to use; and in addition allowing a more specific delivery of heterologous proteins to specific target cells. A further object of the present invention is directed towards the development of a well established classification system in order to determine the prototype strain that can be used to study type III molecules/genes isolated from non-cultivable pathogenic strains.

These aims have been met by following embodiments.

### DETAILED DESCRIPTION OF THE INVENTION

The present invention relates to a safe non-virulent Yersinia enterocolitica mutant strain for delivering heterologous proteins in target cells carrying mutations in at least one of the effector genes yopH, yopO, yopP, yopE, yopM, yopT genes and at least one additional mutation in the invasin genes chosen from yadA and/or inv.

The present invention relates to the production of a disarmed vector Yersinia strain which is totally non-pathogenic and safe to use for medical purposes but still carrying the capacity of transferring heterologous proteins into target cells. The term “Yersinia” as used herein means all species of Yersinia, including Yersinia enterocolitica, Yersinia pseudotuberculosis and Yersinia pestis.

The Yop virulon of Y. enterocolitica encodes six Yop effectors—YopH, YopO/YpkA YopP/YopJ, YopE, YopM and YopT. As described above, functional effector proteins induce a range of modifications to the normal processes of the eukaryotic cells and are toxic to the target cell. A “functional effector protein” refers to an effector protein having a defined catalytic activity and which is capable of eliciting specific toxicity toward the target cells. When using Yersinia as a vector organism to deliver heterologous proteins in eukaryotic cells, especially for medical use, it is of primary importance that all toxic elements are eliminated. Therefore the present inventors suggest to mutate some, preferentially all the genes that code for the six effector proteins, this, in addition to the mutation of one of the invasin genes.

A preferred mutant Yersinia strain of the present invention is a multiple-mutant Yersinia strain in which all the effector-encoding genes are mutated such that the resulting Yersinia no longer produce any functional effector proteins, this, in addition to the mutation of one of the invasin genes. Therefore present invention also relates to a safe non-virulent Yersinia enterocolitica mutant strain for delivering heterologous proteins in target cells carrying mutations in all effector genes yopH, yopO, yopP, yopE, yopM, yopT genes and at least one additional mutation in the invasin genes chosen from yadA and/or inv. Such multiple-mutant Yersinia strain is designated as yopHOPEMTinv or yopHOPEMTyad for Y. enterocolitica; yopHAJEMTinv or yopHAJEMTyad for Y. pseudotuberculosis. With “yopHOPEMT” is meant a strain that is mutated in the effector genes YopH, YopO, YopP, YopE, YopM and YopT. Inv and yad reflects the mutations present in the Inv or YadA genes respectively.

One example of such a yopHOPEMTinv strain is Y. enterocolitica APB40 (pIML421). This strain was deposited at the Belgian Coordinated Collection of Microrganisms—BCCM/LMG Collection (Laboratorium for Microbiology, K. L. Ledeganckstraat 35, B-9000 Gent, Belgium) under deposit number MLG P-21014 on 24 Sep. 2001 by Prof. G. Cornelis (Unité de Pathologie Microbienne (MIPA), Université Catholíque de Louvain, Av. Hippocrate 74, B-1200 Brussels, Belgium). Plasmid pIML421 was generated by crossing serially into the wild-type virulence plasmid pYV40 the following mutations: yopHΔ1-352, yopOΔ65-558, yopP23, yopE21, yopM23, yopT135. In these mutation, the numbers indicate the number of codons that are either left or are deleted if the number is preceeded by the Δ symbol. All these mutations have been crossed using mutator plasmids derived from the suicide vector pKNG101 (streptomycin resistant) and containing the relevant mutations. The mutators are pAB31 (yopHΔ1-352), pAB25 (yopOΔ65-558), pMSK7 (yopP23), pPW52 (yopE21), pAB38 (yopM23), pIML200 (yopT135). To generate the inv mutant APB40(pIML421), the inv allele was disrupted on the chromosome of E40(pIML421) by insertion of the inv mutator pMS154. pMS154 is a derivative of our suicide plasmid pKNG160 (tetracycline resistant) in which we cloned a 1400-bp internal fragment of inv isolated from plasmid pINT192, received from Virginia Miller (University of California, Los Angeles).

Another example of is Y. enterocolitica MRS40 (pIML421). This strain was deposited at the Belgian Coordinated Collection of Microrganisms—BCCM/LMG Collection (Laboratorium for Microbiology, K. L. Ledeganckstraat 35, B9000 Gent, Belgium) under deposit number MLG P-21013 on 24 Sep. 2001 by Prof. G. Cornelis (Unité de Pathologie Microbienne (MIPA), Université Catholique de Louvain, Av. Hippocrate 74, B-1200 Brussels, Belgium).

Yersinia enterocoitica strains produce an enterotoxin called Yst (Delor and Cornelis, 1990). This enterotoxin is encoded by the yst gene present on the chromosome. A strain producing an enterotoxin would not be acceptable for use in patients. Hence, the yst gene has to be removed by using the mutator pID101 which replaces the yst gene by a gene encoding the luciferase from Vibrio harveyi (Delor and Cornelis, 1992).

Under some circumstances, it may be desired to mutate only some but not all of the genes described here before. Indeed, if a strain is too severely disarmed, it might be eliminated from the organism before it made its effect. Accordingly, the present invention further contemplates polymutant Yersinia other than sixtuple-mutant Yersinia, e.g., double-mutant, triple-mutant, quadruple-mutant, quintuple-mutant and sixtuple-mutant Yersinia. Also here the nomenclature only refers to the number of effector genes which are affected.

Possible mutation combinations are: YopHinv, YopHyad, YopHinvyad, YopOinv, YopOyad, YopOinvyad, YopPinv, YopPyad, YopPinvyad, YopEinv, YopEyad, YopEinvyad, YopMinv, YopMyad, YopMinvyad, YopTinv, YopTyad, YopTinvyad, YopHOinv, YopHOyad, YopHOinvyad, YopHPinv, YopHPyad, YopHPinvyad, YopHEinv, YopHEyad, YopHEinvyad, YopHMinv, YopHMyad, YopHMinvyad, YopHTinv, YopHTyad, YopHTinvyad, YopOPinv, YopOPyad, YopOPinvyad, YopOEinv, YopOEyad, YopOEinvyad, YopOMinv, YopOMyad, YopOMinvyad, YopOTinv, YopOTyad, YopOTinvyad, YopPEinv, YopPEyad, YopPEinvyad, YopPMinv, YopPMyad, YopPMinvyad, YopPTinv, YopPTyad, YopPTinvyad, YopEMinv, YopEMyad, YopEMinvyad, YopETinv, YopETyad, YopETinvyad, YopMTinv, YopMTyad, YopMTinvyad, YopHOPinv, YopHOPyad, YopHOPinvyad, YopHOEinv, YopHOEyad, YopHOEinvyad, YopHOMinv, YopHOMyad, YopHOMinvyad, YopHOTinv, YopHOTyad, YopHOTinvyad, YopHPEinv, YopHPEyad, YopHPEinvyad, YopHPMinv, YopHPMyad, YopHPMinvyad, YopHPTinv, YopHPTyad, YopHPTinvyad, YopHEMinv, YopHEMyad, YopHEMinvyad, YopHETinv, YopHETyad, YopHETinvyad, YopHMTinv, YopHMTyad, YopHMTinvyad, YopOPEinv, YopOPEyad, YopOPEinvyad, YopOPMinv, YopOpMyad, YopOPMinvyad, YopOPTinv, YopOPTyad, YopOPTinvyad, YopPEMinv, YopPEMyad, YopPEMinvyad, YopPETinv, YopPETyad, YopPETinvyad, YopEMTinv, YopEMTyad, YopEMTinvyad, YopOETinv, YopOETyad, YopOETnvyad, YopOEMinv, YopOEMyad, YopOEMinvyad, YopOMTinv, YopOMTyad, YopOMTinvyad, YopHOPEinv, YopHOPEyad, YopHOPEinvyad, YopOPEMinv, YopOPEMyad, YopOPEMinvyad, YopPEMTinv, YopPEMTyad, YopPEMTinvyad, YopHPEMinv, YopHPEMyad, YopHPEMinvyad, YopHEMTinv, YopHEMTyad, YopHEMTinvyad, YopOEMTinv, YopOEMTyad, YopOEMTinvyad, YopHOEMinv, YopHOEMyad, YopHOEMinvyad, YopHOETinv, YopHOETyad, YopHOETinvyad, YopHOMTinv, YopHOMTyad, YopHOMTinvyad, YopOPETinv, YopOPETyad, YopOPETinvyad, YopOPMTinv, YopOPMTyad, YopOPMTinvyad, YopHOPMinv, YopHOPMyad, YopHOPMinvyad, YopHOPTinv, YopHOPTyad, YopHOPTinvyad, YopHPETinv, YopHPETyad, YopHPETinvyad, YopHPMTinv, YopHPMTyad, YopHPMTinvyad, YopHOPEMinv, YopHOPEMyad, YopHOPEMinvyad, YopHOPETinv, YopHOPETyad, YopHOPETinvyad YopHOPMTinv, YopHOPMTyad, YopHOPMTinvyad YopHOEMTinv, YopHOEMTyad, YopHOEMTinvyad YopHPEMTinv, YopHPEMTyad, YopHPEMTinvyad YopOPEMTinv, YopOPEMTyad, YopOPEMTinvyad, YopHOPEMTinv, YopHOPEMTyad and YopHOPEMTinvyad.

According to the present invention the mutant strain is used to deliver heterologous proteins into target cells. It was shown that deletion of translocators (YopB, YopD and LcrV) resulted in a translocation-deficient strain (Boyd et al. 2000). Therefore the present inventors suggest to keep these genes active. Also the control elements and the ysc genes which are devoted to the delivery of the Yop proteins are essential in the delivery system, these are preferentially kept functionally active in the mutant vector strain.

For the purpose of the present invention, the terms “mutant strain”, “vector strain” or “mutant vector strain” refer to the strain as such carrying the different mutations as described. The term “recombinant Yersinia”, or “recombinant strain” or “recombinant vector strain” used herein refers to the mutant Yersinia genetically transformed with the expression vectors of the present invention. “Eukaryoic cells” as used herein, the surface to which Yersinia adhere to, are also referred to as “target cells” or “target eukaryotic cells”. In some cases, target cells have a more limited definition, meaning the cells that are specifically recognized by the vector cells. It is clear from the content of the sentence when this latter definition is used. With “specifically” it is implied that there is substantially no cross-interaction of the vector strains with other non-target cells. With “target cell” is meant all cells which are able to interact with type III carrying bacteria and able to accept foreign proteins from this organism.

It was shown that in order for Yersinia species to translocate Yops into eukaryotic cells the bacteria must adhere to the surface of eukaryotic cells. The attachment induces opening of the Ysc secretion channel and increased production of the Yops. The two major determinants of adhesion in Y. pseudotuberculosis and Y. enterocolitica are Invasin and YadA. In addition, in Y. enterocolitica YopN, TyeA and/or LcrG have the potential to be the bacterial ligand necessary to interact with a receptor present on the eukaryotic surface. Nevertheless, it has been recently shown that in Y enterocoitica even such a complex system does not allow selective translocation in target cells (Boyd et al. (2000)). Although it is evident that these Inv and Yad molecules are essential for the transfer of Yop effectors to target cells, the inventors suggest to tackle this problem by eliminating these genes. The inventors found that translocation can be made more specific so that only target cells (eg. phagocytic cells or cancer cells) are targeted. A first step in this approach is to eliminate the elements which trigger this aspecific interaction. The inventors proved that invasin and/or YadA, two powerful adhesins were required for translocation of Yop into non-phagocytic cells. The inventors found that by eliminating the Inv and YadA this aspecific interaction could be abolished without interfering with the transfer capacity of this vector strain to phagocytic cells. If one targets non-phagocytic cells, it is necessary to provide Yersinia with an specific adhesin that recognizes the cells to be targetted. In addition, the inventors found that deletion of the two invasin genes (yadA and inv) avoids invasion at the level of the Peyers patches. This will limit the sphere of action of the vector to the lumen of the intestinal tractas used herein, the “secretion” of a protein refers to the transportation of such protein outward across the cell membrane of a Yersinia. The “translocation” of a protein refers to the transportation of such protein across the plasma membrane of an eukaryotic cell into the cytosol of such eukaryotic cell.

The inventors found that the combination of YopH, YopE and YopT are necessary for the dynamics of phagocytosis by macrophages in vitro.

Six effector genes have been cloned from Y. enterocolitica which are YopE, YopH, YopO, YopM, YopT and YopP. The equivalent effector genes have been cloned from Y. pseudotuberculosis and are named as YopE, YopH, YpkA, YopM, and YopJ, respectively. Some effector genes have also been cloned from Y. pestis. The nucleic acid sequences of these Yop genes are available to those skilled in the art, e.g., in the Genebank Database.

For the purpose of the present invention, the effector-encoding genes are denoted by italicized letters to be distinguished from the effector proteins. Letters of lower case that are italicized denote mutant effector genes. For example, YopE refers to the effector protein encoded by the yopE gene. yopE+ represents the wild type gene, while yopE represents a gene having a mutation.

According to the present invention, a mutant Yersinia strain can be generated by introducing at least one mutation into at least one effector-encoding gene, this combined with at least one mutation in one of the invasin genes. Preferably, such effector-encoding genes include YopE, YopH, YopO/YpkA, YopM, YopT and YopP/YopJ. The skilled artisan may employ any number of standard techniques to generate mutations in these Yop genes. Sambrook et al. describe in general such techniques. See Sambrook et al. (1989) Molecular Cloning: A Laboratory Manual, Second Edition Cold Spring Harbor Laboratory Press: Cold Spring Harbor, N.Y. The present invention illustrates that a safe non-virulent Yersinia enterocolitica mutant strain for delivering heterologous proteins in target cells may contain (a) mutation(s) located in the noncoding sequence of said gene and/or in the coding sequence of said gene.

The term “mutation” is used herein as a general term and includes changes of both single base pair and multiple base pairs. Such mutations may include substitutions, frame-shift mutations, deletions, truncations, insertions, duplications or any modification in the nucleic acid encoding said polypeptide, or at a different location in the genome of said cells, influencing the expression of said nucleic acid or polypeptide. In case point mutations occur, the number of nucleotides will be identical compared to the original sequence; only a change in nucleotide sequence can be observed. This stands in contrast with the other listed mutations where the number of the nucleotides will be different from the number observed in the wild type sequence and consequently will also reflect in a change of the nucleotide sequence.

In accordance with the present invention, the mutation can be generated in the promoter region of an effector-encoding gene so that the expression of such effector gene is abolished.

The mutation can also be generated in the coding region of an effector-encoding gene such that the catalytic activity of the encoded effector protein is abolished. The “catalytic activity” of an effector protein refers to the anti-target cell function of an effector protein, i.e., toxicity. Such activity is governed by the catalytic motifs in the catalytic domain of an effector protein. The approaches for identifying the catalytic domain and/or the catalytic motifs of an effector protein are well within the knowledge of those skilled in the art. See, for example, Sory et al. (1995), Boland et al. (1996) and Cornelis et al. (1997).

Accordingly, one preferred mutation of the present invention is a deletion of the entire catalytic domain. Another preferred mutation is a frameshift mutation in an effector-encoding gene such that the catalytic domain is not present in the protein product expressed from such “frameshifted” gene. A most preferred mutation is a mutation with the deletion of the entire coding region.

Other mutations are also contemplated by the present invention, such as small deletions or base pair substitutions, which are generated in the catalytic motifs of an effector protein leading to destruction of the catalytic activity of a given effector protein.

The mutations that are generated in the Yop genes may be introduced into Yersinia by a number of methods. One such method involves cloning a mutated Yop gene (i.e., a yop gene), into a “suicide” vector which is capable of introducing the mutated yop sequence into Yersinia via allelic exchange. Such “suicide” vectors are described by Kaniga et al. (1991) Gene 109: 137-141 and by Sarker et al. (1997) Mol. Microbiol 23: 409-411.

In this manner, mutations generated in multiple Yop genes may be introduced successively into Yersinia, giving rise to polymutant recombinant Yersinia. The order in which these mutated yop sequences are introduced is not important.

In this way, a mutant disarmed Y.enterocolitica strain is created which is safe to be used for medical use causing no infection in the patient.

Additional mutations can be introduced in order to guarantee that the mutant Yersinia vector strain is safe to use. Therefore the present invention also relates to the optional mutation onto the previously described mutation of YopR, YopO, YlpA, YomA, Yst, β-Lac or any gene linked to the iron acquisition system.

YopR (encoded by the yscH gene) and YopQ (encoded by yopQ are transported by the type III system but no clear functions have been assigned for both proteins yet. It has been suggested for YopQ that it may play a role in regulation the size of the pore made by the type III system. The fact that both proteins are conserved within the type III system suggests their role in virulence. In addition, as both proteins use the transport system they may compete with the transport of the heterologous protein. Therefore, the present inventors suggest to mutate both yscH and yopQ genes (Boyd et al. 2000).

In the mouse model, there is no clear difference in the virulence between wild-type W22703 and an ylpA mutant called W22708 (pYL4). However, this observation is of little value because W22703 is of very low virulence for the mouse and hence the model does not allow to detect minor differences. The inventors have shown that the expression of lipoprotein YlpA (China et al., 1990) is regulated by the same regulator that regulates yop, ysc and yadA genes. Thus, YlpA is expressed at the bacterial surface only at 37° C. Moreover, YlpA is conserved among all the pathogenic strains. For these reeasons, the inventors consider that this outer membrane protein must play a role in virulence.

YomA (Iriarte,Mand Cornelis, G. R., 1999) is an putative outer membrane protein with unknown function and conserved over diverse type III systems. The expression of YomA is also regulated like that of the Yops, YadA and YlpA. Moreover, it is conserved among the pathogenic strains. Because of this, the inventors suggest it must have a role in virulence. Mutants are presently beeing engineered and their phenotype will be characterized with respect to animal virulence, adherence and proteins translocation.

The present invention also suggests to delete the chromosome gene encoding the thermostable enterotoxin Yst (Delor and Cornelis, 1992). As this is a toxin it is preferably removed from the mutant vector strain.

Y. enterocolitica carries on its chromosome two endogenous β-Lactamase genes, β-lactamase A and β-I actamase B (Cornelis and Abraham, 1974). β-Lactamase A is essentially active against ampicillin while β-lactamase B is a cephalosporinase. Removal of β-LactamaseA gene makes this vector strain sensitive to the common antibiotic ampicillin and equivalent antibiotics. Especially when using a strain for medical purpose, it is of prime importance that it can be readily eliminated. Therefore, the present invention also suggests to eliminate β-Lactamase A by mutation, using the mutator plasmid pKNG105 (Kaniga, Delor and Cornelis, 1991). As an example, strain MRS40 is the blaA mutant of strain E40.

The present invention also suggests to devoid the mutant vector strain of any iron acquisition system. Y. enterocohitica is then dependent from iron supplemented in the medium. As iron is limited in body fluids, growth in vivo of this strain is limited. This contributes to the production of a much safer and well-considered vector strain. Strains of serotype O :9, like E40 or W22703 described here, are naturally devoid of an iron chelating system.

The delivery vector plasmid contains the 5′ end of a yop gene followed by a restriction site, so as to allow in frame cloning of genes of interest. With delivery vector, or expression vector is meant the DNA fragment that carries the heterologous gene allowing its expression in the vector strain.

A further aspect of the present invention is directed to an expression vector for use in combination with the a mutant Yersinia strain according to the present invention to deliver a desired protein into eukaryotic cells. In accordance with the present invention, such a vector is characterized by (in the 5′ to 3′ direction) a promoter, a first nucleic acid sequence encoding a delivery signal from a Yersinia effector protein, operably linked to said promoter, a second nucleic acid sequence fused thereto coding for a heterologous protein to be delivered.

In accordance with the present invention, the promoter of the expression vector is preferably derived from a Yersinia virulon gene. A “Yersinia virulon gene” refers to genes on the Yersinia pYV plasmid, the expression of which is controlled both by temperature and by contact with a target cell. See review by Cornelis et al. (1997). Such genes include genes coding for elements of the secretion machinery (the Ysc genes), genes coding for translocators (YopB, YopD, and LcrV), genes coding for the control elements (YopN and LcrG), and genes coding for effectors (YopE, YopH, YopO/YpkA, YopM and YopP/YopJ).

In a preferred embodiment of the present invention, the promoter is from an effector-encoding gene selected from any one of YopE, YopH, YopO/YpkA, YopM and YopP/YopJ. More preferably, the promoter is from YopE.

Further in accordance with the present invention, a first DNA sequence coding for a delivery signal is operably linked to the promoter.

“A delivery signal”, as described herein above, refers to a polypeptide which can be recognized by the secretion and translocation system of Yersinia and therefore directs the secretion and translocation of a protein into a target cell.

The term “delivery” used herein refers to the transportation of a protein from a Yersinia to a eukaryotic cell, including the steps of expressing the protein in the Yersinia, secreting the expressed protein(s) from such Yersinia and translocating the secreted protein(s) by such Yersinia into the cytosol of the eukaryotic cell.

According to the present invention, such a “delivery” polypeptide is derived from an effector protein. The effector proteins include YopE, YopH, YopO/YpkA, YopM, and YopP/YopJ. Preferably, the effector protein is YopE. More preferably, the effector protein is YopE of Yersinia enterocolitica

One skilled in the art is familiar with the methods for identifying the polypeptide sequences of an effector protein that are capable of delivering a protein. For example, one such method is described by Sory et al. (1994). Briefly, polypeptide sequences from various portions of the Yop proteins can be fused in-frame to a reporter enzyme such as the calmodulin-activated adenylate cyclase domain (or Cya) of the Bordetella pertussis cyclolysin. Delivery of a Yop-Cya hybrid protein into the cytosol of eukaryotic cells is indicated by the appearance of cyclase activity in the infected eukaryotic cells that leads to the accumulation of cAMP. This accumulation can be measured and gives an indication upon efficiency of the delivery signal. Examples of such delivery signal polypeptides include from Y. enterocolitica. YopE130 (the N-terminal 130 amino acids of YopE), YopE50, YopM100 and YopH71.

By employing such an approach, one skilled in the art can determine, if desired, the minimal sequence requirement, i.e., a contiguous amino acid sequence of the shortest length, that is capable of delivering a protein. See, e.g., Sory et al. (1994). Accordingly, preferred delivery signals of the present invention consists of at least the minimal sequence of amino acids of a Yop effector protein that is capable of delivering a protein. Preferably, such a fragment consists of 15 to 50, preferably 20, 25, 30, 35, 40 contiguous amino acids of a protein.

Further in accordance with the present invention, a second DNA sequence encoding a heterologous protein is fused in frame to the first DNA sequence in the instant vector for delivery into eukaryotic cells. The term “heterologous protein” used herein refers to a protein other than a Yersinia Yop protein. “Yop proteins” refer to Yersinia virulon proteins that are secreted, including the translocators and the effectors.

According to the present invention, “a heterologous protein” includes naturally occurring proteins or parts thereof. The term “part of a protein” includes a peptide or polypeptide fragment of a protein that is of sufficient length to carry an activity. Preferably, such a fragment consists of at least 8 or 9 contiguous amino acids of a protein. The present invention illustrates that said heterologous protein is a protein that is naturally occurring or a protein which is encoded by a gene but whereby the protein has never been demonstrated in nature. With “a protein which is encoded by a gene but whereby the protein has never been demonstrated in nature” is meant that a protein/peptide can be made from a synthetic gene, whereby the protein/peptide may have a structure similar or non-similar to natural proteins/peptides. Proteins/peptides which are similar to natural proteins/peptides may be derivatives or variants of natural proteins; or may be structural analogues of organic structures. These heterologous entities are condemned to be peptidic structures as they need to be expressed from DNA coding sequences. This “non-natural protein” also includes artificially engineered proteins or parts thereof, such as fusion of two or more naturally occurring proteins or parts thereof, polypeptides (in-frame fusion of two or more peptide epitopes) as exemplified by Thompson et al. (1995) in Proc. Natl. Acad. Sol. USA 92: 5845-5849.

The protein expressed from the fused first and second DNA sequences is also termed as a “fusion protein” or a “hybrid protein”, i.e., a hybrid of Yersinia delivery signal and a heterologous protein.

There is no particular limitation in the heterologous protein that can be delivered. The present invention particularly contemplates proteins, such as, e.g., tumor associated proteins, antigens of pathogens, killer agents and anti-inflammatory compounds. According to the present invention, said heterologous protein, produced upon recombinant expression, is an antigen, a toxin or a drug.

In a preferred embodiment of the present invention, said heterologous protein is an antigen or at least one epitope of said protein whereby the antigen (epitope) is (is derived from) a tumor associated protein (TAA), an infection-related protein.

A “tumor associated protein” refers to a protein that is specifically expressed in tumors or expressed at an abnormal level in tumors relative to normal tissues. Such tumor associated proteins include, but are not limited to, members of the MAGE family, the BAGE family (such as BAGE-1), the DAGE/Prame family (such as DAGE-1), the GAGE family, the RAGE family (such as RAGE-1), the SMAGE family, NAG, Tyrosinase, Melan-A/MART-1, gp100, MUC-1, TAG-72, CA125, mutated proto-oncogenes such as p21ras, mutated tumor suppressor genes such as p53, tumor associated viral antigens (e.g., HPV16 E7), HOM-MEL-40, HOM-MEL-55, NY-COL-2, HOM-HD-397, HOM-RCC-1.14, HOM-HD-21, HOM-NSCLC-11, HOM-M EL-2.4, and HOM-TES-11. Members of the MAGE family include, but are not limited to, MAGE-1, MAGE-2, MAGE-11. Members of the GAGE family include, but are not limited to, GAGE-1, GAGE-6. See, e.g., review by Van den Eynde and van der Bruggen (1997) in Curr. Opin. Immunol. 9:684-693, Sahin et al. (1997) in Curr. Opin. Immunol. 9:709-716, and Shawler et al. (1997). These proteins have been shown to associate with certain tumors such as melanoma, lung cancer, prostate cancer, breast cancer, renal cancer and others.

A number of known antigens derived from pathogens can also be employed according to the present invention and is called “an infection-related protein”. Pathogens contemplated by the present invention include a parasite, a yeast, a fungi, a bacterium or a virus. Specific examples of antigens characteristic of a pathogen include the influenza virus nucleoprotein (residues 218-226, as set forth in F. et al. (1997) J. Virol. 71: 2715-2721) antigens from Sendal virus and lymphocytic choriomeningitis virus (see, An et al. (1997) J. Virol. 71: 2292-2302), the B1 protein of hepatitis C virus (Bruna-Romero et al. (1997) Hepatology 25: 470-477), the virus envelope glycoprotein gp 160 of HIV (Achour et al. (1996) J. Virol. 70: 6741-6750), amino acids 252-260 or the circumsporozite protein of Plasmodium berghei (Allsopp et al. (1996) Eur. J. Immunol. 26: 1951-1958), the influenza A virus nucleoprotein (residues 366-374, Nomura et al. (1996) J. Immunol. Methods 193: 4149), the listeriolysin O protein of Listera monocytogenes (residues 91-99, An et al. (1996) Infect. Immun. 64: 1685-1693), the E6 protein (residues 131-140, Gao et al. (1995) J. Immunol. 155: 5519-5526) and E7 protein (residues 21-28 and 48-55, Bauer et al. (1995) Scand. J. Immunol. 42: 317-323) of human papillomavirus type 16, the M2 protein of respiratory syncytial virus (residues 82-90 and 81-95, Hsu et al. (1995) Immunology 85: 347-350), the herpes simplex virus type 1 ribonucleotide reductase (see, Salvucci et al. (1995) J. Gen. Virol. 69:1122-1131) and the rotavirus VP7 protein (see, Franco et al. (1993) J. Gen. Virol. 74: 2579-2586), P. falciparum antigens (causing malaria) and hepatitis B surface antigen (Gilbert et al. (1997) Nature Biotech. 15:1280-1283).

Accordingly, sequences coding for the above-described proteins may be cloned into the present expression vector for delivery.

A number of coding sequences for small antigenic peptides can also be employed in the present invention. One skilled in the art can readily determine the length of the fragments required to produce immunogenic peptides. Alternatively, the skilled artisan can also use coding sequences for peptides that are known to elicit specific T cell responses, such as tumor-associated antigenic peptides (TRAs) as disclosed by U.S. Pat. No. 5,965,381.

Antigenic peptides of a pathogen origin can also be used, such as those disclosed by Gilbert et al. (1997).

In the present invention said heterologous gene may code for a protein comprising the active subunit of a toxin whereby said toxin is chosen from the group comprising the diphteria toxin (dtxA), the cholera toxin (A1) and the antraxtoxin (LF and EF).

The diphteria toxin consists of two subunits, subunit A and subunit B. The fragment A of the diphteria toxin inhibits protein synthesis by ADP-ribosylation of elongation factor 2. It is normally delivered into eukaryotic cells by fragment B. Without fragment B, it is non-toxic, unless it is delivered into cells by another means. The inventors expressed this dtxA gene and proved that Yersinia can transfer this heterologous protein into several eukaryotic cell types (Boyd et al. 2000).

The cholera toxin is an oligomeric protein with a mass of 85,620 Daltons, with a total of 755 amino acid residues. The toxin is made of an A-subunit and 5 identical B subunits. This toxin binds to ganglioside G_{M1} receptors in the small intestine. It is then cleaved, and the enzymatic subunit enters the cell leading to a rise of intracellular cAMP. This results in the hyperactivity of Na⁺ pumps, leading to diarrhea. When the A-subunit of the cholera toxin is inserted into the cell is cleaved along a disulfide bridge between A1 and A2, which keeps the protein inactive. (Merritt 1994). The A1 subunit of cholera toxin is the enzymatically active portion of the protein molecule, and it acts as an ADP-ribosyltransferase. Ribosylation of Gs stabilizes the GTP bound form of the protein, lower its GTPase activity creating a near constitutively on signal for the generation of adenylyl cyclase, and therefore elevating cAMP levels. In the present invention, the inventors suggest to use the A1 subunit of the CTX to kill target cells using the Yersinia type III system.

Virulent strains of Bacillus anthracis produce three distinct antigenic components related to a complex exotoxin called the anthrax toxin. Each component of the toxin is a thermolabile protein with a MW of approximately 80 kDa. Factor I is the edema factor (EF) which is necessary for the edema producing activity of the toxin. EF is known to be an inherent adenylate cyclase, similar to the Bordetella pertussis adenylate cyclase toxin. Factor II is the protective antigen (PA) or binding (B) domain of the anthrax toxin carrying the two active (A) domains, EF (above) and LF (below). Factor III is known as the lethal factor (LF) because it is essential for the lethal effects of the anthrax toxin. Apart from their antigenicity, each of the three factors separate exhibits no significant biological activity in an animal. However, combinations of two or three of the toxin components yield the following results in experimental animals: PA+LF combine to produce lethal activity, EF+PA produce edema and PA+LF+EF produces edema and necrosis and is lethal. EF+PA has been shown to elevate cyclic AMP to extraordinary levels in susceptible cells. Changes in intracellular cAMP are known to affect changes in membrane permeability and may account for edema. The present inventors suggest the use of LF and/or EF in combination with a Yersinia vector strain according to the present invention. PA is a carrying molecule and is not needed when using an alternative delivery system such as described Yersinia system.

Alternatively, said heterologous protein of the present invention is a drug. According to a preferred embodiment of the present invention, said drug is an anti-inflammatory compound. Preferentially, according to the present invention said anti-inflammatory compound is selected from the group comprising yopP or any intracellular compound inhibiting the NFkB-CREB pathway.

YopJ of Yersinia pseudotuberculosis inhibits multiple signal transduction pathways that converge to induce phosphorylation of the transcrition factor CREB (cAMP response element-binding protein). This transcription factor is intimately linked to cell fate in many different tissues. Meijer et al. (2000) propose YopJ as an antagonist circumventing innate and adaptive immune response at multiple levels. It was suggested to provide new avenues for therapeutic intervention in diseases where there is a need to block chronic inflammatory diseases. YopP is the YopJ homologue in Y. enterocolotica. The present inventors have shown that when one of the proteins is injected into animal cells strongly induces anti-inflammatory reaction; this protein prevents the release of cytokine tumor necrosis factor alpha (TNFα), a cytokine that is central in the onset of the inflammatory response (BOLAND, A. and CORNELIS, G. R. Role of YopP in suppression of TNFα release by macrophages during Yersinia infection. Infection and Immunity, 1998, 66:1878-1884).

The present inventors suggest according to the present invention that live Y.enterocolitica, disarmed from all the Yop effectors but overproducing YopP could be used to treat inflammatory diseases.

The inhibition of NFκB activation is accompanied by a lack of activation of the mitogen-activated protein (MAP) kinases (MAPKs), c-Jun-N-terminal kinase (JNK), p38, and extracellular signal regulated kinase (ERK) 1 and 2 that is observed upon infection of macrophages by Yersinia producing YopP. Lack of activation of these MAPKs results from the inhibition of the upstream MAPK kinases (MAPKKs) by binding of YopP (Cornelis 2000). According to the present invention, said intracellular compound inhibiting the NFκB-CREB pathway may be chosen from the group comprising IκB and MAPKK-, MAPK-, JNK-, p38-, or ERK-inhibitors.

One preferred embodiment of the present invention is directed to a Yersinia of the above-described mutant Yersinia strain transformed with an expression vector for delivery of a heterologous protein as described above into a target cell.

Sequences coding for a full-length naturally occurring protein, a part of a naturally occurring protein, combinations of parts of a naturally occurring protein, or combinations of different naturally occurring proteins or parts from different proteins, may all be employed in the present invention. For example, a sequence coding for multiple functions may be used.

Those skilled in the art are familiar with the techniques to make DNA fragments coding for a part of a protein, or link a DNA sequence encoding a part of one protein in frame to a DNA sequence encoding a part of another protein and the like.

The vectors of the present invention may include other sequence elements such as a 3′ termination sequence (including a stop codon and a poly A sequence), or a gene conferring a drug resistance which allows the selection of Yersinia transformants having received a vector according to the present invention.

The expression vectors of the present invention may be transformed by a number of known methods into Yersinia. For the purpose of the present invention, the methods of transformation for introducing an expression vector include, but are not limited to, electroporation, calcium phosphate mediated transformation, conjugation, or combinations thereof. For example, a vector can be transformed into a first bacteria strain by a standard electroporation procedure. Subsequently, such a vector can be transferred from the first bacteria strain into Yersinia by conjugation, a process also called “mobilization”. Yersinia transformant (i.e., Yersinia having taken up the vector) may be selected, e.g., with antibiotics. These techniques are well known in the art. See, for example, Sory et at. (1994). Nevertheless, after the strain has been selected, the antibiotic resistance needs to be removed this to keep the Yersinia strict to the definition of a safe vector strain as defined by the present invention.

Accordingly, the present invention contemplates a method for delivering heterologous proteins as herein above described into eukaryotic cells.

By “target”, is meant the extracellular adhesion of Yersinia to an eukaryotic cell. The present invention contemplates a wide range of eukaryotic cells that may be targeted by the instant recombinant Yersinia.

As described herein above, a first step in creating a non-pathogenic, non-adherent strain according to the present invention is to eliminate invasin and/or YadA which normally trigger interaction with target cells. This result in a strain that is not able to transfer any protein to any target cells anymore. The present invention further suggest that in a second step said Y. enterocolitica polymutant can be targeted specifically to target cells by labeling these cells with a specific targeting signal. Therefore, the present invention relates in particular to a safe non-virulent Yersinia enterocolitica mutant strain carrying mutation in at least one of the effector genes (YopH, yopO, yopP, yopE, yopM, yopT), in at least one additional mutation in the Invasin genes (yadA, inv) and which is specifically modified with a targeting signal allowing a cell specific interaction of said Yersinia strain with a specific cell type. The present invention also gives some evidence that addition of the targeting signal ‘as such’ without additional deletion of the invasin genes improves already major the specific delivery of proteins into target cells.

Therefore present invention relates generally to a Yersinia enterocolitica mutant strain which is specifically modified with a targeting signal allowing a cell specific interaction of said Yersinia strain with a specific cell type.

According to the present invention said targeting signal, which is carried by a Yersinia enterocolitica mutant according to the present invention, may be a protein, a peptide, a lipid or a combination thereof. According to the present invention, said targeting signal is carried by a bacterial surface display system. There are a variety of bacterial surface display systems for peptides. These can be the presented outside the cell via pili, autotransporters, lpp-ompA and LamB. Some of these surface display systems have proved useful for controlled bacterial adhesion (eg. type I pili and lpp-ompA) to inorganic and organic surfaces (such us ZnO and cellulose).

According to the present invention said displayed peptides may be scFv and small protein domains.

Some scFv and protein domains show high affinity to surface markers found in the target cell. Efficiency of recognition between vector cell and target cells is determined by the interaction efficiency of both compounds, the peptide exposed by the recombinant vector strain and the receptor thereof present on the target cell. The higher the efficiency, the faster the interaction will occur and the lower the percentage of non-specific interactions will be. The virulon system is dependent on this contact and/or regulated under specific promoters activated by contact- or intracellular bacterial growth as described for the virulon system of type III bacteria.

The inventors determined that antibodies recognizing the cell markers could be engineered onto the surface delivery system of in Yersinia resulting in a high affinity antigen-antibody reaction; proving that the adherence is strong enough to promote delivery. In this study the inventors analysed the transfer of YopE or Cya. YopE disrupts actin filaments and provokes a very clear cytopathic effect which is easy to monitor; transfer of the Cya reporter results in an accumulation of cyclic AMP which can be assayed by a routine, commercial ELISA test in the target cell allowing quantification of the translocation.

According to the present invention, said targeting signal carried by a Yersinia enterocolitica mutant according to the present invention strain may be a bacterial adhesin. In a preferred embodiment of the present invention said bacterial adhesin is selected from the group comprising Opa-proteins and Afa-proteins.

Neisseria meningitidis shows a specific predilection for binding to the leptomeninges and meningeal blood vessels in human brain and not to the cerebral cortex. The major ligand that mediates adherence is the pilus. The presence of Opa protein increases the association of Cap+ meningococci that expressed low-adhesive pili, but do not influence the association of high-adhesive pili (Hardy et al. 2000) Mol Microbiol 2000 May;36(4):817-29). A number of neisserial adhesins (i.e. pilli, Opa, Opc and P36) and additional putative virulence determinants which affect bacterial adherence and invasion into host cells (i.e. LOS, capsule, PorB) have been identified. Clearly, neisserial surface variation serves as an adaptive mechanism which can modulate tissue tropism, immune evasion and survival in the changing host environment. Important progress has been made in recent years with respect to the host cellular receptors and subsequent signal transduction processes which are involved in neisserial adherence, invasion and transcytosis. This has led to the identification of (i) CD46 as a receptor for pilus which allows adherence to epithelial and endothelial cells, (ii) HSPGs, in cooperation with vitronectin and fibronectin, as receptors for a particular subset of Opa proteins and Opc, which may both mediate invasion into most epithelial and endothelial cells, and (iii) CD66 as the receptors for most Opa variants, potentially being involved in cellular interactions including adherence, invasion and transcytosis with epithelial, endothelial and phagocytic cells. (reviewed by Dehio et al. Subcell Biochem 2000;33:61-96. Therefore, the present inventors suggest to use this Opa protein as targeting protein on a mutant recombinant vector strain to target epithelial, endothelial and phagocytic cells.

The afimbrial adhesive sheath of Escherichia coli, encoded by the afa-3 gene cluster, is composed of two proteins with different roles in bacterium-HeLa cell interactions. AfaE is required for adhesion and AfaD for internalization. AfaD is the prototype of a family of invasins encoded by adhesion-associated operons in pathogenic E. coli. (Garcia et al FEBS Lett 2000 August 18;479). The present inventors suggest to use this AfaE protein as targeting protein on a mutant recombinant vector strain to target human intestinal cells cells, since this adhesins recognizes the brush-border-associated decay-accelerating factor (DAF; CD55).

Alternatively, the Yersinia enterocolitica mutant strain according to the present invention may carry a specific targeting signal chosen from antibodies able to recognize a specific cell marker.

According to a preferred embodiment of the invention, said cell specific marker recognized by the targeted Yersinia enterocolitica mutant strain is selected from the group comprising members of tumor-antigen, parasite-specific antigen. Examples of said proteins are given in previous paragraphs. Cell surface markers of human colon cancer, at early and late stages, have been identified. For instance, colonic cancer cells could be targeted with antibodies directed against specific surface antigens such as Ep-CAM, carcinoembryogenic antigen (CEA), STEAP or A33.

Further the present invention relates to a Yersinia enterocolitica mutant strain as defined above (see any of claims 14 to 20) and comprising mutations as defined above (see any of claims 1 to 4).

The present invention further contemplates a method for delivering a heterologous protein into a target cell comprising contacting said target cell with a Yersinia mutant strain according to the present invention.

Preferably, the method according to the present invention allows the delivery of a heterologous protein into a target cell wherein said target cell is an eukaryotic cell of plant, human, animal or parasitic origin. Further in accordance with the present invention, the delivery of a protein can be achieved by contacting an eukaryotic cell with a recombinant Yersinia under appropriate conditions. Various references and techniques are conventionally available for those skilled in the art regarding the conditions for inducing the expression and translocation of virulon genes, including the desired temperature, Ca++ concentration, manners in which Yersinia and target cells are mixed, and the like. See, for example, Cornelis, Cross talk between Yersinia and eukaryotic cells, Society for General Microbiology Symposium, 55; MoCRAE, SAUNDERS, SMYTH, STOW (eds), Molecular aspects of host-pathogen interactions, Cambridge University Press, 1997. The conditions may vary depending on the type of eukaryotic cells to be targeted, e.g., the conditions for targeting human epithelial carcinoma Hela cells (Sory et al. (1994)); the conditions for targeting mouse thymoma or melanoma cells (Starnbach et al. (1994) J. Immunol. 153: 1603); the conditions for targeting mouse macrophages (Boland et al. (1996)). Such variations can be addressed by those skilled in the art using conventional techniques.

Those skilled in the art can also use a number of assays to determine whether the delivery of a fusion protein is successful. For example, the fusion protein may be labeled with an isotope or an immunofluoresceine, or detected by a immunofluoresceine conjugated antibody, as disclosed by Rosqvist et al. (1994) EMBO J. 13: 964. The determination can also be based on the enzymatic activity of the protein being delivered, e.g., the assay described by Sory et al. (1994). The determination can also be based on the antigenicity of the protein being delivered. For example, the delivery of a MAGE-1 protein into EBV-transformed human B cells can be detected by the recognition of such targeted B cells by CTL cells specific for MAGE-1 epitopes. Such CTL recognition, in turn, may be detected by a number of assays including assaying the secretion of IFN-.gamma. from the activated CTLs or Cr@51 release from lysed target cells. Methods such as Western-blot analysis using antibodies specific against the protein being delivered, PCR in situ hybridization, or ELISPOT (Mabtech A B, Sweden) may also be employed for such determination. See, e.g., W. Herr et al. (1997) J. Immunol. Methods 203: 141-152 and W. Herr et al. (I 996) J. Immunotl Methods 191: 131-142.

More specifically, said eukaryotic target cell may be selected from the group consisting of an antigen presenting cell, a cancer cell, an infected cell or an inflamed cell.

In particular, the present invention contemplates antigen-presenting cells. “Antigen presenting cells” as referred herein express at least one class I or class II MHC determinant and may include those cells which are known as professional antigen-presenting cells such as macrophages, dendritic cells and B cells. Other professional antigen-presenting cells include monocytes, marginal zone Kupffer cells, microglia, Langerhans' cells, interdigitating dendritic cells, follicular dendritic cells, and T cells. Facultative antigen-presenting cells can also be used according to the present invention. Examples of facultative antigen-presenting cells include astrocytes, follicular cells, endothelium and fibroblasts. As used herein, “antigen-presenting cells” encompass both professional and facultative types of antigen-presenting cells.

In a further aspect of the present invention, recombinant Yersinia capable of delivering proteins to antigen-presenting cells are employed for inducing an immune response. Accordingly, the present invention contemplates immunogenic compositions and methods for inducing specific immune responses using the recombinant Yersinia according to the present invention as described herein above.

The immune responses contemplated by the present invention include cellular immune responses (mediated primarily by T cells) and humoral immune responses (mediated primarily by antibodies). Janeway and Travers teach in general these immune response. (Janeway and Travers (1996) Immunology, The Immune System in Health and Disease 2nd ed. Garland Publishing, Inc.: New York, N.Y., and London, England.) (See also, review by O. Tureci et al. (1997) Molecular Medicine Today 3(8): 342-349.

The antigen presenting cells can be isolated from tissue or blood (containing peripheral blood mononuclear cells) samples obtained from a mammal such as a human or rodent. Cell lines established from such samples may also be used. Procedures for establishing cell lines are well known in the art. Certain cell lines may be obtained directly from the American Type Culture Collection, 10801 University Boulevard, Manassas, Va. 20110-2209. Both normal and malignant cells may be employed.

In accordance with a preferred embodiment of the present invention, the MHC determinants expressed by the antigen presenting cell are compatible with those expressed by the mammal involved, and at least one of these MHC determinants is capable of presenting one or more antigenic epitopes derived from the protein being delivered.

One skilled in the art is also familiar with the methods for determining whether the MHC molecules expressed by the antigen presenting cell are compatible with those of the mammal subject involved, such as well known HLA-typing procedures. See general teachings by Coligan et al. (1994) Current Protocols in Immunology John Wiley & Sons Inc: New York, N.Y.

Those skilled in the art are able, through the extensive teachings in the art, to determine the MHC molecule for presentation of a particular antigen. For example, U.S. Pat. No. 5,405,940 teaches the determination of HLA-A1 as the presenting molecule for a peptide of MAGE-1, EADPTGHSY; U.S. Pat. No. 5,558,995 teaches the determination of HLA-Cw1601 for presenting another peptide of MAGE-1, SAYGEPRKL; U.S. Pat. No. 5,530,096 teaches the determination of HLA-A2 as the presenting molecule for a peptide of Tyrosinase, MLLAVLYCL. In the event the eukaryotic cells being targeted do not express a desired HLA or MHC molecule, the gene encoding such molecule may be introduced into the eukaryotic cells by well known transformation or transfection procedures.

With “infected cell” is meant a cell which carries bacteria, parasites, yeast, fungus or a virus; “inflamed cell” indicates that within the cell a inflammatory pathway is activated such as found in autoimmune diseases.

The present invention also relates to a composition for use as a medicament or a cell based product intended for clinical use comprising a Yersinia enterocolitica mutant strain as described above. The pathological conditions contemplated by the present invention which can be targeted by the composition according to the present invention include tumors and Infections by pathogens such as bacteria, parasites, yeast, fungus or virus. By “treating”, is meant alleviating or inhibiting a pathological condition, e.g., inhibiting tumor growth or metastasis, reducing the size of tumor, or diminishing symptoms of a pathogen infection. The recombinant Yersinia of the present invention can also be employed in vivo, i.e., introducing recombinant Yersinia into a mammal, such as a human or rodent subject.

In particular, the present invention also contemplates the use of a composition for the preparation of a medicament for treating cancer, infections and inflammatory diseases. Said inflammatory disease may be an autoimmune diseases. The present invention also provides a method for treatment of cancer, infections and autoimmune diseases comprising the use of said composition.

According to the present invention, said method can be applied for treating cancer comprising administering to a subject in need of treatment a therapeutically effective amount of a composition according to the invention, wherein the antigen is a tumor specific antigen. In this approach the Yersinia vector strain provides antigens to antigen presenting cells allowing the triggering of a T-cell immune response. This allows the activation of a complex machinery resulting in the elimination of the tumor carrying this specific tumor antigen.

A safe recombinant Yersinia may be employed in an immunogenic composition to induce an immune response for treating various pathological conditions in mammals. The pathological conditions contemplated by the present invention include tumors and pathogen infections, as disclosed herein.

Alternatively, said method can also be applied for treating cancer comprising administering to a subject in need of treatment a therapeutically effective amount of a composition according to the present invention, wherein a heterologous toxin is made and delivered to cancer cells. In this approach cancer cells are directly targeted, this in contrast to previous approach whereby the immunological response is responsible for the elimination of the cancer cells. According to the present invention a “killer” Yersinia enterocolitica only adheres to the cancer cells and not to any other cell. Said Y. enterocolitica polymutant, devoid of any effector but producing a lethal toxin, such as the diphteria toxin, can be used to approach and kill specific cancer cells. In addition, said method can be applied for treating infections comprising administrating to a person in need of treatment a therapeutically effective amount of a composition according to the present invention, wherein the antigen is an infectious specific antigen. T-cells can be triggered by antigen presenting cells carrying the specific antigen provided by the vector strain of the present invention. In this way infected cells can be recognized by the immune response and eliminated. Alternatively, said method can be used for treating infections comprising administering to a person in need of treatment a therapeutically effective amount of a composition according to the present invention, wherein a heterologous toxin is made and delivered to said infected cells. This is an alternative approach compared to previous method. In this method the infected cells are targeted directly and does not need the activation of the immune system. When the vector strain expresses (a) toxin(s), said method allows the killing of a target cell comprising contacting the target cell with said Yersinia enterocolitica mutant strain or a composition. Here the target cell may be a cancer cell, an infected cell, an activated cell of the immunological system (autoimmune diseases).

The method according to the present invention also provides the possibility to treat autoimmune disease comprising administering to a subject in need of treatment a therapeutically effective amount of a composition according to the present invention, wherein the antigen is a self protein linked to a MHC-factor. In this way, immune cells which are presenting self-proteins are eliminated, resulting in the decrease of the autoimmune response. Self-proteins as such are not recognized. Alternatively, the method according to the present invention can be used to treat inflammatory diseases comprising administering to a person in need of treatment a therapeutically effective amount of a composition according to the present invention, wherein an anti-inflammatory compound or toxin is expressed in the vector strain and delivered to said inflamed cells. Here also, the targeting is direct and allows the direct elimination of inflamed cells. The anti-inflammatory compound used is yopP or an intracellular compound inhibiting the NFkB-CREB pathway. In this way, the pathway, which normally results in the activation of the immune cells, is inhibited.

Until now, the composition according to the present invention is used to have a direct effect on the diseased state. Alternatively, this composition can also be used as a vaccine adjuvant. In this approach, the main compound that activates the immune system is different from the composition of the present invention; this latter is added to have a cooperative effect on the first. This vaccine adjuvant comprises a composition according to the present invention. The present invention also discloses the method for immunizing against a disease in humans or animals comprising administering a vaccine comprising an adjuvant of the present invention.

A “vaccine” is an immunogenic composition capable of eliciting protection against infections, whether partial or complete. A vaccine may also be useful for treatment of an individual, in which case it is called a therapeutic vaccine. Said vaccine compositions may include prophylactic as well as therapeutic vaccine compositions. The term “therapeutic” refers to a composition capable of treating infections. “Prophylactic” refers to a composition for treating patients which are susceptible to infections in order to prevent coming infection.

The present invention further contemplates the administration of the composition of the present invention in the subject. This may be carried out orally or parenterally such as topical (including ophthalmic), intraperitoneal, subcutaneous, intradermal, intrapeural, intrathecal, intramuscular, intralymphoidal or intratumoral administration.

For in vivo use of recombinant Yersinia, the safety can be tested in animals beforehand. In this case, the recombinant Yersinia may be administered to the animal orally or directly into the stomach. The animals may be sacrificed a few days (1-3 days) after the administration of the recombinant Yersinia The intestines are washed and the Peyer patches or the faeces can be examined for viable Yersinia. See, e.g., Sory et al. (1992) Infect. Immun. 60: 3830-3836. The recombinant Yersinia may also be administered to the animal by intraperitoneal injection. Organs of sacrificed animals such as spleen and liver can be examined for the presence of intracellular Yersinia, an indication of insufficient safety. Intracellular Yersinia may be detected by e.g., cultivating cell extracts on solid medium. See teachings by Sory et al. (1988) Microb. Pathogen 4: 431-442.

Formulations for such administrations may comprise an effective amount of the composition according to the present invention in physiological saline or any buffer/media deprived from any possible allergen. When applied orally, the vector strain will remain in the digestive tract. This application therefore is focused on the cure of diseases located within the digestive tract. Preferentially, said part of the digestive tract is the colon. One of the frequently occurring autoimmune diseases of the digestive tract in humans is the Crohne disease.

The present invention also relates to the pharmacological composition comprising the compound according to the invention and optionally a pharmaceutical acceptable carrier, diluent or excipient. The immunogenic compositions can include, in addition to a recombinant Yersinia, other substances such as cytokines, adjuvants and pharmaceutically acceptable carriers. Cytokines can also be included in such immunogenic compositions using additional recombinant Yersinia of the present invention capable of delivering a cytokine, for example.

These compositions may, for example, be administered parentally or intravenously. The compositions according to the, invention for parenteral administration can be, in particular, sterile solutions, aqueous or non-aqueous, suspensions or emulsions, As a pharmaceutically acceptable solution or vehicle propylene glycol, polyethylene glycol, injectable organic esters, for example ethyl oleate, or cyclodextrins may be employed. These compositions can also comprise wetting, emulsifying and/or dispersing agents.

They may also be prepared in the form of sterile solid compositions which may be dissolved at the time of use in sterile water or any other sterile injectable medium.

The present invention can also comprise adjuvants which are well known to a person skilled in the art (vitamin C, antioxidant agents etc.) capable of being used in synergy with the compounds according to the invention in order to improve and prolong the treatments of the diseases.

Compositions can be provided together with physiologically tolerable liquid, gel or solid carriers, diluents, adjuvants and excepients. Formulations may contain such normally employed additives as binders, fillers, carriers, preservatives, stabilizing agents, emulsifiers, buffers and excipients as for example, pharmaceutical grades of mannitol, lactose, starch, magnesium stearate, sodium saccharin, cellulose, magnesium carbonate and the like. These compositions contain 1%-95% of active ingredient, preferably 2%-70%.

The compositions are preferentially prepared as injectables, either as liquid solutions or suspensions; solid forms suitable for solution in, or in suspension in, liquid prior to injection may also be prepared.

The composition of the present invention are often mixed with diluents or excipients which are compatible and physiologically tolerable. Suitable diluents and exipients are for example, water, saline, dextrose, glycerol, or the like, and combinations thereof. In addition, if desired the compositions may contain minor amounts of auxiliary substances such as wetting or emulsifying, stabilizing or pH buffering agents.

The present invention also relates to a method to produce a safe non-virulent Yersinia enterocolitica mutant strain for delivering heterologous proteins in target cells comprising the transformation of polymutant cells carrying mutations in at least one of the effector genes (yopH, yopO, yopP, yopE, yopM, yopT) and at least one additional mutation in the invasin genes (yadA and/or inv) with an expression vector as defined by the present invention.

As described herein above, recombinant vector strains can be made by introducing expression vector in the corresponding vector strain. This final vector strain can be applied to deliver heterologous proteins/DNA in a large variety of cells. According to the present invention, the production of said strain can occur in vitro or in vivo. Most probably, the recombinant strain will be made in vitro as described above, but it is not excluded that specific phages carrying the desired expression vector can be injected together or separate from the target strain. This specific phage can infect the vector strain within the body of the subject.

The present invention also suggests the use of a Yersinia enterocolitica mutant strain according to the invention for the preparation of in vitro screening assays. Said in vitro screening method using a Yersinia enterocolitica mutant can be used in different respects.

Said method can be applied for detecting T cell mediated activity of a target antigens peptide, comprising at least the following steps:

(a) providing a Yersinia enterocolitica mutant strain according to the invention carrying an antigenic peptide,

(b) contacting an antigen presenting cell with said Yersinia mutant strain, thereby providing an activated T cell,

(c) contacting a target cell with said activated T cell, and,

(d) monitoring the effect of said activated T cell on said target cell, thereby detecting is anti-target activity.

In the design and conduct of above described applications, important considerations include methods for introducing the antigen into MHC class I and II processing pathways, methods for selecting the correct vector strain, route of administration and antigen selection. Because the cell therapy as presented in the present invention needs a specific recognition of the target cell, it is important that indeed the choice of antigen is well considered. Therefore the present invention suggests that the antigen is a tumor specific antigen, an infectious specific antigen or a self-protein-MHC-complex when applied in the treatment of cancer, infections (viral, bacterial, parasitical) or autoimmune diseases. In addition, it is important that the compositions are administered to a person in need of treatment in a therapeutically effective amount. Example of antigens that might be considered as tumor antigens are described by Fong and Engleman 2000³⁶.

Alternatively said method can be applied for detecting peptides or proteins interfering with the NFkB/CREB pathway, comprising at least the following steps: (a) providing a Yersinia enterocolitica mutant strain according to the invention carrying an peptide/protein peptide as agonist or antagonist candidate for the NFkB pathway, (b) contacting an target cell which has been activated using LPS or an alternative thereof with the recombinant vector strain. It is known that LPS activates the NFkB pathway, and, (c) monitoring the effect of said vector strain on said target cell, thereby detecting anti-inflammatory activity. According to this embodiment, the present invention provides a method of identifying compounds (peptides or proteins) which selectively inhibit, induce or interfere with the expression/production of the polypeptides necessary in the NFkB pathway. Compounds may carry agonistic or antagonistic properties.

A cell-mediated immune response allows the natural elimination of specific cells. The specificity depends on the specificity of the antigen that is used to activate the antigen presenting cells. In this approach according to the present invention the antigen is delivered to the antigen presenting cell using a vector strain. Said strain expresses the antigen as heterologous protein. According to the present invention the method for inducing a cell-mediated immune response specific for a heterologous protein can occure in vitro, comprising the steps of: (a) selecting an antigen presenting cell expressing an MHC molecule capable of presenting at least one epitope of said heterologous protein; (b) forming a cell mixture by contacting said antigen presenting cell with a Yersinia enterocolitica mutant strain carrying mutations in at least one of the effector genes (yopH, yopO, yopP, yopE, yopM, yopT) and at least one additional mutation in the invasin genes (yadA and/or inv) and expressing an antigen, and, (c) contacting a sample containing peripheral blood lymphocytes taken from a subject, with the cell mixture formed in step (b) thereby inducing in vitro, a cell-mediated response specific for said heterologous protein. Recombinant Yersinia can also be employed in an ex vivo regime for inducing CTLs specific for a protein. The procedure to develop such specific CTLs in vitro is known in the art, e.g., as disclosed by the U.S. Pat. No. 5,342,774. Briefly, a blood sample containing T cell precursors is taken from a mammal. PBLs are purified from such blood sample and are incubated with stimulator cells expressing an antigenic epitope in the context of an MHC molecule. CTLs specific for such epitope produced can be detected by assays such as an assay for Cr@51 release or secretion of IFN-gamma.

According to the present invention, a mixture of a recombinant Yersinia and an antigen presenting cell can be used as the “stimulator cell” in such an in vitro procedure for producing CTLs specific for the protein being delivered. The MHC determinants expressed by the antigen presenting cell used are compatible with those expressed by the mammal from which PBLs are isolated, and at least one of these MHC molecules is capable of presenting, to T cells, one or more epitopes derived from the protein being delivered. CTL cells generated as such can be administered, in a therapy regimen of adoptive transfer, to a mammal a pathological condition characterized by an abnormal expression of the protein used in the delivery system. See teachings by Greenberg (1986) J. Immunol. 136 (5): 1917; Riddel et al. (1992) Science 257: 238; Lynch et al. (1991) Eur. J. Immunol. 21: 1403; and Kast et al. (1989) Cell 59: 603 for adoptive transfer. CTLs, by lysing the cells abnormally expressing such antigens, can alleviate or treat the pathological condition at issue such as a tumor, an infection with a parasite or a virus.

Alternatively, the method for inducing a cell-mediated immune response specific for a heterologous protein can occur in vivo, comprising the steps of: (a) selecting a Yersinia mutant strain carrying mutations in at least one of the effector genes (yopH, yopO, yopP, yopE, yopM, yopT) and at least one additional mutation in the invasin genes (yadA and/or inv) and expressing an antigen; (b) forming a cell mixture by contacting said Yersinia mutant strain with an antigen presenting, and, (c) contacting peripheral blood lymphocytes with the cell mixture formed in step (b). In said method at least step (a) is performed in vitro; steps (c) and/or (d) may be performed in vivo by injecting the mutant stain (a) or the cell mixture (b) into a subject.

In all these approaches the induction of the immune system is needed. The antigen presenting cells absorb the recombinant Yersinia cells using their natural systems. As the inventors proved that the Yersinia does not have specific interaction with target cells, it might also be important to increase specificity of the Yersinia for the antigen presenting cell. Therefore the present invention also relates to a method where a cell-mediated immune response is elicted using a specific targeting system as described by the invention. In particular this invention relates to a method for inducing in vitro, a cell-mediated immune response specific for a heterologous protein, comprising the steps of: (a) selecting a recombinant Yersinia enterocolitica according to the invention expressing an MHC molecule presenting at least one epitope of a heterologous protein; (b) forming a cell mixture by contacting an antigen presenting cell with said Yersinia enterocolitica mutant strain, and, (c) contacting a sample containing peripheral blood lymphocytes taken from a subject, with the cell mixture formed in step (b) thereby inducing in vitro, a cell-mediated response specific for said heterologous protein.

The present invention also relates to a method for inducing in vivo, a cell-mediated immune response specific for a heterologous protein, comprising the steps of: (a) selecting a recombinant Yersinia enterocolitica according to the present invention expressing an MHC molecule presenting at least one epitope of a heterologous protein; (b) forming a cell mixture by contacting an antigen presenting cell with said Yersinia enterocolitica mutant strain, and, (c) contacting a sample containing peripheral blood lymphocytes taken from a subject, with the cell mixture formed in step (b) thereby inducing in vitro, a cell-mediated response specific for said heterologous protein; whereby at least step (a) is performed in vitro; steps (b) and/or (c) may be performed in vivo by injecting the mutant stain (a) or the cell mixture (b) into a subject.

The present invention also further contemplates a method for monitoring a cellular immune response in a subject before, during and after a vaccination regimen, comprising the steps of: (a) obtaining from said subject an antigen presenting cell expressing an MHC molecule; (b) forming a cell mixture by contacting said antigen presenting cell with a Yersinia strain according to the present invention, wherein said second DNA sequence in the expression vector codes for at least one epitope of said antigen which is presented by said MHC molecule of said antigen presenting cell; thereby delivering said heterologous protein into said antigen presenting cell; and (c) contacting a sample containing peripheral blood lymphocytes taken from said subject, with the cell mixture formed in step (b), and assaying for the presence of a cell-mediated immune response specific for said antigen thereby monitoring a cell-mediated immune response in said subject before, during and after a vaccination regimen. According to this aspect of the present invention, the immune responses induced with the recombinant Yersinia can be utilized in a number of regimes for diagnostic or therapeutic use. For example, recombinant Yersinia can be employed in an in vitro procedure for monitoring the efficacy of a vaccination therapy in a mammal such as a human or rodent. In this regime, certain antigen presenting cells (e.g., dendritic cells) are taken from a subject being vaccinated with immunogenic compositions, e.g., a particular antigen. Such antigen presenting cells are then contacted with recombinant Yersinia capable of delivering the antigen which is used for vaccination. Subsequently, peripheral blood lymphocytes taken from the same subject (i.e., autologous PBLs) are added, preferably in combination with cytokines such as IL-2, to the mixture of antigen presenting cells and Yersinia. The efficacy of the vaccination can be assessed after priming and then after successive boosts by the presence of CTLs or antibodies that are specific for the relevant antigen. The presence of specific CTLs can be detected using standard assays such as an assay for Cr@51 release or for the secretion of IFN-gamma. The presence of specific antibodies can be detected by assays such as ELISA using the antigens which are immobilized on a culture plate, or a standard proliferation assay for T-helper cells.

In addition, the present invention also describes a method for detecting direct anti-target activity (anti-cancer or anti-inflammatory or anti-infectious activity) of a Yersinia enterocolitica mutant strain as described by the invention, comprising at least the following steps: (a) providing a Yersinia enterocolitica mutant strain according to the invention, expressing a protein marker (b) contacting a target (cancer, infected or inflamed) cell with said Yersinia strain, (c) monitoring the effect of said Yersinia strain, on said target cell, thereby detecting anti-target activity.

The present invention also provides a kit for detecting T cell mediated activity of a target antigenic peptide or for detecting direct antitarget activity (anti-cancer or anti-inflammatory or anti-infectious activity), comprising at a Yersinia enterocolitica mutant strain according to the invention.

The present invention also relates to a secretion-type-III-carrying strain chosen from Pseudomonas (P. aeroginosa), Bordetella (B. pertussis), Burholdeda (B. cepacia), Chlamidia strains or Yersinia (Y. enterocoitica, Y. pseudotuberculosis, or Y. pestis) carrying polymutations in equivalent genes as described for Y. enterocolitica carrying mutations in at least one of the effector genes (yopH, yopO, yopP, yopE, yopM, yopT) and at least one additional mutation in the invasin genes (yadA, inv).

The present invention also relates to an expression vector for delivering a heterologous protein into a eukaryotic cell using a secretion-type III-carrying strain carrying mutations in at least one of the equivalent effector genes (yopH, yopO, yopP, yopE, yopM, yopT) and at least one additional mutation in the equivalent invasin genes (yadA, inv) carrying elements as defined hertein above wherein said Yersinia sequence elements are exchanged by a sequence element or signal specific for the strain used or compatible therewith.

The present invention also relates to a secretion type III carrying bacterial strains according to present invention transformed with an expression vector according to present invention; Its use for the delivery of heterologous proteins in a target cell as described herein for Y enterocolitica; a composition as described for Y enterocolitica according to present invention; its use for the preparation of a medicament usable in the treatment of various diseases as described for Y enterocolitica according to present invention, or its use in in vitro tests as described for Y enterocolitica according to present invention.

### BRIEF DESCRIPTION OF THE FIGURE AND TABLE

FIG. 1: Effect of YopB, YopE, YopH, YopT on phagocytosis

Table 1: List of the strains and genotype.

### MODES FOR CARRYING OUT THE INVENTION

### EXAMPLE 1

### Role of YopT and Other Yops in Prevention of Phagocytosis Bacterial Strains, Plasmids and Growth Conditions

The bacterial strains and plasmids used in this study are listed in Table I. The strains were routinely grown in tryptic soy broth (TSB) and plated on tryptic soy agar (TSA) containing the required antibiotics. For in vitro induction of the yop genes, Y. enterocolitica was grown in brain heart infusion (BHI), supplemented with 20 mM sodium oxalate, 20 mM MgCl₂ and 0.4% glucose (BHI-ox). Prior to cell infection, Y. enterocolitica was grown in non-supplemented BHI. Selective agents were used at the following concentrations: ampicillin 200 μg/ml, chloramphenicol 10 μg/ml, nalidixic acid 35 μg/ml, streptomycin 100 μg/ml, kanamycin 50 μg/ml, sucrose 5% and 1 mM arsenite.

Molecular Biology Techniques

Yops were precipitated from culture supernatants by ammonium sulfate (400 mg/ml) (Cornelis et al., 1987), analysed by SDS-PAGE and where appropriate transfered to nitrocellulose membranes immunoblots were developed using a secondary antibody conjugated to horse-radish peroxidase and Supersignal (Pierce, Rockford, Ill., USA) as chemiluminescent substrate. Mutator plasmids were introduced into Y. enterocolitica strains by conjugation from E. coli SM10λpir. After selection on appropriate media the mutations were confirmed by PCR analysis and by Yop induction and SDS-PAGE analysis of the secreted proteins or Western blot analysis of the bacterial proteins.

Infection Procedures

J774 macrophages were aliquoted into 24-well plates the day before infection. On the day of infection the cells were washed with fresh culture medium. Y. enterocolitice strains were grown overnight in TSB at 22° C. They were inoculated in BHI at OD600=0.2, grown for 2 h at 22° C. and pre-induced by incubating the cells during 30 min at 37° C. After harvesting and washing in prewarmed saline, the bacteria were added to the cells (moi depends on assay). The infections were carried out for 30 min at 37°. Then the cells were washed 3 times in culture medium after infection before proceeding.

Determination of Bacterial Uptake by Immunofluorescence

To distinguish between intra- end extracellularly located bacteria, the double-immunofluorecence test described by Heesemann and Laufs (1984) was used. The presence of intracellular and extracellular bacteria was studied. When high percentages of extracellular bacteria were present indicated that phagocytosis was inhibited.

Briefly, 30 min after infection the cell cultures were washed and the cover slips were overlaid with a rabbit antiserum directed against yersinia LPS, diluted (1/500) in phosphate-buffered saline (PBS) and incubated for 30 min at 4° C. These antibodies only bind to extracellular bacteria. Monolayers were washed four times with PBS and fixed with ice-cold methanol for 90 s. After methanol removal, extracellular bacteria were labeled with Texas-red-conjugated goat anti-rabbit antiserum (1 g/ml) for 20 min at 37° C., followed by four washes in PBS. Both intra- and extracellular bacteria were then stained by incubation of cover slips for 1 h at 37° C. with anti-LPS antiserum, washed in PBS and treated with fluorescein isothiocyanate-conjugated goat anti-rabbit antiserum. After four washes in PBS, cover slips were mounted in Mowiol mounting medium on a glass slide.

Extracellular bacteria were detected by exitation at 596 nm (Texas-red) and total cell-associated bacteria were detected at 490 nm (FITC). For each experiment, 50 cells per cover slips were randomly selected, and the numbers of extracellular and total cell-associated bacteria were determined. The experiments have been done three times in duplicate.

Result of Phacytosis Assay with J774 Macrophages Cell Line

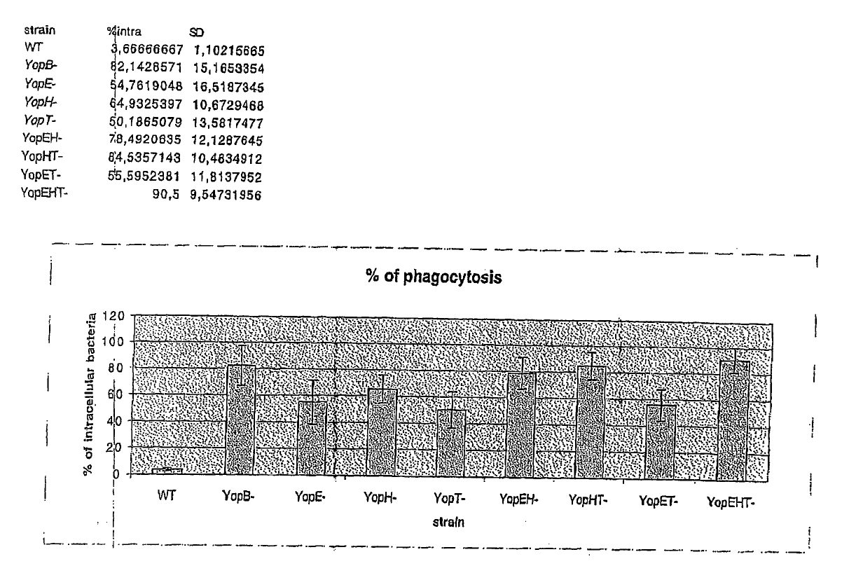

To determine the potential roles of different Yersinia enterocolitica gene products in mediating phagocytosis, several Y.enterocolitica mutants, were examined by immunofluorescence (see FIG. 1),

The virulence plasmid-containing wild-type strain MRS 40 (pYV40) markedly inhibited the phagocytic capacity of the J774 cells. Of all the bacteria that became associated with the cells, more than 90% remained extracellularly bound. For the yopB-strain, unable to inject the different effector proteins in eukaryotic cells, only 18% were located outside. The ability of the yopH deletion mutant MRS40 (pSI4008) to inhibit phagocytosis was greatly reduced, less than 36% remained extracellular. Similar results were obtained for the yopE mutated strain MRS40 (pAB4052) where less than 45% remained extracellularly bound. The present inventors showed thar YopT, a 35-kDa protein, which is involved in cytoskeleton alteration and actin filament structure disruption (Iriarte and Cornelis, 1998) is also involved in antiphagocytic effect of Yersinia. Indeed, 50% of associated bacteria are phagocytosed in a yopT deletion mutant MRS40 (pIM409).

When the inventors studied the phagocytosis of double mutant of these three genes they observed each time that the phagocytosis percentage is higher than for the single mutant and lower than the triple mutant (data not shown).

Hence, YopH YopE and YopT are involved in phagocytosis resistance to macrophages but not sufficient to have alone a total antiphagocytic effect.

The foregoing example is illustrative of the invention and is not intended to limit the scope of the invention as set out in the claims. All of the references cited herein are incorporated by reference.

References

Meijer et al. (2000) The bacterial protein YopJ abrogates multiple signal transduction pathways that converge on the transcription factor CREB. Cell. Micribiol. 2: 231-238.

Boland, A., Cornelis, G. R. (1998): Role of YopP in suppression of tumor necrosis factor alpha release by macrophages during Yersinia infection. Infect. Immun. 66, 1878-1884.

Boland, A., Sory, M. P., Iriarte, M., Kerbourch, C., Wattiau, P., Cornelis, G. R. (1996): Status of YopM and YopN in the Yersinia Yop virulon: YopM of Y. enterocolitica is internalized inside the cytosol of PU5-1.8 macrophages by the YopB, D, N delivery apparatus. EMBO J. 15, 5191-5201.

Cornelis, G. R., Vanooteghem, J. C., Sluiters, C. (1987): Transcription of the yop regulon from Y. enterocolitica requires trans acting pYV and chromosomal genes. Microb. Pathog. 2, 367-379.

Heesemann, J., and Laufs. (1985): Double immunofluorescence microscopic technique for accurate differentiation of extracellularly and intracellularly located bacteria in cell culture. J. Clin. Microbiol; 22,168-175.

Iriarte, M., Cornelis, G. R. (1998a): YopT, a new Yersinia Yop effector protein, affects the cytoskeleton of host cells. Mol. Microbiol. 29, 915-929.

Mills, S. D., Boland, A., Sory, M. P., Van Der Smissen, P., Kerbourch, C., Finlay, B. B., Cornelis, G. R. (1997): YopP, a novel Yersinia effector protein, is delivered into macrophages to induce apoptosis. Proc. Natl. Acad. Sci. USA 94, 12638-12643.

Sarker, M. R., Neyt, C., Stainier, I., Cornelis, G. R. (1998a): The Yersinia Yop virulon: LcrV is required for extrusion of the translocators YopB and YopD. J Bacteriol 180, 1207-1214.

Sambrook, J., Fritsch, E. F., Maniatis, T. (1989): Molecular Cloning: a laboratory manual, 2nd ed. Cold Spring Harbor Laboratory Press, Cold Spring Harbor, N.Y.

TABLE 1
List of the strains and genotypes

| Bacterial strains | genotype | references |
|---|---|---|
| MRS40(pAB404) | yopE₂₁, yopH_{Δ1-352} | Boland and Cornelis, |
| | | 1998 |
| MRS40(pIM424) | yopE₂₁, yopT₁₃₅ | Iriarte and Cornelis, |
| | | 1998 |
| MRS40(pIM425) | yopH_{Δ1-352}, yopT₁₃₅ | Boyd et al., 2000 |
| MRS40(pIM426) | yopE₂₁, yopH_{Δ1-352}, | Boyd et al., 2000 |
| | yopT₁₃₅ | |
| MRS40(pPW401) | yopB_{Δ89-217} | Boland et al., 1996 |
| MRS40(pSI4008) | yopH_{Δ1-352} | Mills et al., 1997 |
| MRS40(pYV40) | wild type | Sarker et al., 1998a |
| MRS40(pAB4052) | yopE₂₁ | Boland and Cornelis, |
| | | 1998 |
| MRS40(pIM409) | yopT₁₃₅ | Iriarte and Cornelis, |
| | | 1998 |

1. A safe non-virulent Yersinia enterocolitica mutant strain for delivering heterologous proteins in target cells carrying mutations in at least one of the effector genes yopH, yopO, yopP, yopE, yopM, yopT genes and at least one additional mutation in the invasin genes chosen from yadA and/or inv.

2. A safe non-virulent Yersinia enterocolitica mutant strain for delivering heterologous proteins in target cells according to claim 1 carrying mutations in all effector genes yopH, yopO, yopP, yopE, yopM, yopT genes and at least one additional mutation in the invasin genes chosen from yadA and/or inv.

3. A safe non-virulent Yersinia enterocolitica mutant strain for delivering heterologous proteins in target cells according to claims 1 or 2 further comprising an optional mutation present in at least one of the genes chosen from YopR, YopQ, YlpA, YomA, Yst, β-Lac or any gene linked to the iron acquisition system

4. A safe non-virulent Yersinia enterocolitica mutant strain for delivering heterologous proteins in target cells according to any of the claims 1 to 3 wherein said mutation is a mutation of the non-coding sequence of said gene and/or a mutation of the coding sequence of said gene.

5. An expression vector for delivering a heterologous protein into a target cell using a Yersinia enterocolitica mutant strain according to any of the claims 1 to 4, which comprises in the 5′ to 3′ direction:
(a) a promoter of a Yersinia virulon gene,
(b) a first DNA sequence encoding a delivery signal from a Yersinia effector protein, operably linked to said promoter; and,
(c) a second DNA sequence coding for said heterologous protein, fused in frame to the 3′ end of said first DNA sequence.

6. An expression vector according to claim 5 wherein said heterologous protein is a protein that is naturally occurring or a protein which is encoded by a gene but whereby the protein has never been demonstrated in nature.

7. An expression vector according to claim 6 wherein said heterologous protein, produced upon recombinant expression, is an antigen, a toxin or a drug.

8. An expression vector according to claim 7 wherein said heterologous protein is an antigen or at least one epitope of said protein is, whereby the antigen (epitope) is (is derived from) a tumor associated protein (TAA), an infection-related protein.

9. An expression vector according to claim 7 wherein said heterologous protein comprises the active subunit of a toxin, wherein said toxin is chosen from the group comprising the diphteria toxin (dtxA), the cholera toxin (A1) and the antraxtoxin (LF and EF).

10. An expression vector according to claim 7 wherein said drug (peptide/protein) is an anti-inflammatory compound.

11. An expression vector according, to claim 10 wherein said anti-inflammatory compound is selected from the group consisting of yopP or an intracellular compound inhibiting the NFkB-CREB pathway.

12. An expression vector according to claim 11 wherein said intracellular compound inhibiting the NFkB-CREB pathway is chosen from the group comprising IκB and MAPKK-, MAPK-, JNK-, p38-, or ERK-inhibitors.

13. A Yersinia enterocolitica mutant strain according to any of the claims 1 to 4 transformed with an expression vector according to any of the claims 5 to 12.

14. A Yersinia enterocolitica mutant strain which is specifically modified with a targeting signal allowing a cell specific interaction of said Yersinia strain with a specific cell type.

15. A Yersinia enterocolitica mutant strain according to claim 14 wherein said targeting signal is a protein, a peptide, a lipid or a combination thereof.

16. A Yersinia enterocolitica mutant strain according to claim 14 wherein said targeting signal is carried by a bacterial surface display system.

17. A Yersinia enterocolitica mutant strain according to claim 15 wherein said targeting protein is a bacterial adhesin.

18. A Yersinia enterocolitica mutant strain according to claim 17 wherein said bacterial adhesin is selected from the group comprising OPA-proteins, AFA-proteins.

19. A Yersinia enterocolitica mutant strain according to claim 15 wherein said protein is an antibody recognizing a specific cell marker.

20. A Yersinia enterocolitica mutant strain according to claim 19 wherein said cell specific marker is selected from the group comprising a tumor-antigen or parasite-specific antigen.

21. A Yersinia enterocolitica mutant strain according to claim 14 to 20 comprising mutations as defined in any one of claims 1 to 4.

22. A method for delivering a heterologous protein into a target cell, comprising contacting said target cell with a Yersinia mutant strain according to any of the claims 1 to 4 or 13 to 21.

23. A method for delivering a heterologous protein into a target cell according to any of the claims 1 to 21 wherein said target cell is an eukaryotic cell of plant, human, animal or parasitic origin.

24. A method according to claim 23 wherein said eukaryotic target cell is selected from the group consisting of an antigen presenting cell, a cancer cell, an infected cell and an inflamed cell.

25. A method according to claim 24 wherein said antigen presenting cell is selected from the group consisting of a B cell, a macrophage, a dendritic cell, a monocyte, a follicular cell and a fibroblast.

26. A composition for use as a medicament or a cell based product intended for clinical use comprising a Yersinia enterocolitica mutant strain according to any of the claims 1 to 4 and 13 to 21.

27. A pharmaceutical composition comprising the compound according to claim 26 and optionally a pharmaceutical acceptable carrier, diluent or excipient.

28. Use of a composition according to claims 26 or 27 for the preparation of a medicament for treating cancer, infections and inflammatory diseases.

29. Use of a composition according to claims 26 or 27 wherein said inflammatory disease is an autoimmune disease.

30. Use of a composition according to claim 26 or 27 as a vaccine adjuvant.

31. Vaccine adjuvant comprising a composition according to claim 26 or 27.

32. A method for immunizing against a disease in humans or animals comprising administering a vaccine comprising an adjuvant of claim 31.

33. A method for treatment of cancer, infections or autoimmune diseases comprising the use of a composition according to claim 26 or 27.

34. A method for treating cancer comprising administering to a person in need of treatment a therapeutically effective amount of a composition of claim 26 or 27, wherein the antigen as defined in claim 20 is a tumor specific antigen.

35. A method for treating cancer comprising administering to a person in need of treatment a therapeutically effective amount of a composition of claim 26 or 27, wherein a heterologous toxin is made and delivered to cancer cells.

36. A method for treating infections comprising administering to a person in need of treatment a therapeutically effective amount of a composition of claim 26 or 27, wherein the antigen is an infectious specific antigen.

37. A method for treating infections comprising administering to a person in need of treatment a therapeutically effective amount of a composition of claim 26 or 27, wherein a heterologous toxin is made and delivered to said infected cells.

38. A method for treating autoimmune disease comprising administering to a person in need of treatment a therapeutically effective amount of a composition of claim 26 or 27, wherein the antigen is a selfprotein coupled to an MHC-factor.

39. A method for treating inflammatory diseases comprising administering to a person in need of treatment a therapeutically effective amount of a composition of claim 26 or 27, wherein an anti-inflammatory compound or toxin is made and delivered to said inflamed cells.

40. A method according to any of the claims 32 to 39 wherein said disease is located within the digestive tract.

41. A method according to claim 40 wherein said part of the digestive tract is the colon.

42. A method according to claim 41 wherein said disease is the Crohne disease.

43. A method of treatment according to any of claims 32 to 42, whereby the administration of the composition according to claim 26 or 27 in the patient is carried out orally and/or parenterally; whereby parenterally administration includes topical (including ophthalmic), intraperitoneal, subcutaneous, intradermal, intrapeural, intrathecal, intramuscular, intralymphoidal and/or intratumoral administration.

44. A method to produce a Yersinia enterocolitica mutant strain comprising the transformation of a polymutant strain according to any of the claims 1 to 4 with an expression vector as defined in claims 5 to 12.

45. A method for delivering a heterologous protein into a cell, whereby the production of a strain as described in claim 44 occurs in vitro or in vivo.

46. A method for killing a target cell comprising contacting said target cell with a Yersinia enterocolitica mutant strain or a composition according to claim 26 or 27.

47. Use of a Yersinia enterocolitica mutant strain according to any of claims 1 to 4 and 13 to 21 for the preparation of in vitro screening assays.

48. An in vitro screening method using a Yersinia enterocolitica mutant strain according to any of the claims 1 to 4 and 13 to 21.

49. A method for detecting T cell mediated activity of a target antigenic peptide, comprising at least the following steps:
(a) providing a Yersinia enterocolitica mutant strain according to any of the claims 13 to 21 carrying an antigenic peptide,
(b) contacting an antigen presenting cell with said Yersinia mutant strain,
(c) contacting a target cell with said activated T cell, and,
monitoring the effect of said activated T cell on said target cell, thereby detecting anti-target activity.

50. A method for detecting peptides or proteins interfering with the NFkB/CREB pathway, comprising at least the following steps:
(a) providing a Yersinia enterocolitica mutant strain according to any of the claims 13 to 21 carrying an peptide/protein peptide as agonist or antagonist candidate for the NFkB pathway,
(b) contacting an target cell which has been activated using LPS or an alternative thereof with the recombinant vector strain, and,
(c) monitoring the effect of said vector strain on said target cell, thereby detecting anti-inflammatory activity.

51. A method for inducing in vitro, a cell-mediated immune response specific for a heterologous protein, comprising the steps of:
(a) selecting an antigen presenting cell expressing an MHC molecule capable of presenting at least one epitope of said heterologous protein,
(b) forming a cell mixture by contacting said antigen presenting cell with a Yersinia enterocolitica mutant strain according to any of the claims 13 to 21 expressing an antigen, and,
(c) contacting a sample containing peripheral blood lymphocytes taken from a subject, with the cell mixture formed in step (b) thereby inducing in vitro, a cell-mediated response specific for said heterologous protein.

52. A method for inducing in vivo, a cell-mediated immune response specific for a heterologous protein, comprising the steps of:
(a) selecting a Yersinia mutant strain according to any of the claims 13 to 21 expressing an antigen,
(b) forming a cell mixture by contacting said Yersinia mutant strain with an antigen presenting, and,
(c) contacting peripheral blood lymphocytes with the cell mixture formed in step (b)
whereby at least step (a) is performed in vitro; steps (c) and/or (d) may be performed in vivo by Injecting the mutant stain (a) or the cell mixture (b) into a subject.

53. A method for inducing in vitro, a cell-mediated immune response specific for a heterologous protein, comprising the steps of:
(a) selecting a recombinant Yersinia enterocolitica according to any of the claims 13 to 21 expressing an MHC molecule presenting at least one epitope of a heterologous protein,
(b) forming a cell mixture by contacting an antigen presenting cell with said Yersinia enterocolitica mutant strain, and,
(c) contacting a sample containing peripheral blood lymphocytes taken from a subject, with the cell mixture formed in step (b) thereby inducing in vitro, a cell-mediated response specific for said heterologous protein.

54. A method for inducing in viva, a cell-mediated immune response specific for a heterologous protein, comprising the steps of:
(a) selecting a recombinant Yersinia enterocolitica according to any of the claims 13 to 21 expressing an MHC molecule presenting at least one epitope of a heterologous protein,
(b) forming a cell mixture by contacting an antigen presenting cell with said Yersinia enterocolitica mutant strain, and,
(c) contacting a sample containing peripheral blood lymphocytes taken from a subject, with the cell mixture formed in step (b) thereby inducing in vitro, a cell-mediated response specific for said heterologous protein,
wherein at least step (a) is performed in vitro; and wherein steps (b) and/or (c) may be performed in vivo by injecting the mutant stain (a) or the cell mixture (b) into a subject.

55. A method for monitoring a cellular immune response in a subject before, during and after a vaccination regimen, comprising the steps of:
(a) obtaining from said subject an antigen presenting cell expressing an MHC molecule,
(b) forming a cell mixture by contacting said antigen presenting cell with a Yersinia strain according to claims 13 to 21, wherein said second DNA sequence in the expression vector codes for at least one epitope of said antigen which is presented by said MHC molecule of said antigen presenting cell; thereby delivering said heterologous protein into said antigen presenting cell, and,
(c) contacting a sample containing peripheral blood lymphocytes taken from said subject, with the cell mixture formed in step (b), and assaying for the presence of a cell-mediated immune response specific for said antigen thereby monitoring a cell-mediated immune response in said subject before, during and after a vaccination regimen.

56. A method for detecting direct anti-target activity (anti-cancer or anti-inflammatory or anti-infectious activity) of a Yersinia enterocolitica mutant strain as described in any of the claims 13 to 21, comprising at least the following steps:
(a) providing a Yersinia enterocolitica mutant strain according to any of the claims 23 to 28, expressing a protein marker,
(b) contacting a target cell with said Yersinia strain, and,
(c) monitoring the effect of said Yersinia strain, on said target cell, thereby detecting anti-target activity.

57. A kit for detecting T cell mediated activity of a target antigenic peptide or for detecting direct anti-target activity, comprising at a Yersinia enterocolitica mutant strain according to any of claims 1 to 4 and 13 to 21.

58. A secretion-type-III-carrying strain chosen from Pseudomonas (P. aeroginosa), Bordetella (B. pertussis), Burholderia (B. cepacia), Chlamidia strains or Yersinia (Y. enterocolitica, Y. pseudotuberculosis, or Y. pestis) carrying polymutations in equivalent genes as described for Y. enterocolitica according to any of the claims 1 to 4.

59. An expression vector for delivering a heterologous protein into a eukaryotic cell using a secretion-type III-carrying strain according to claim 58 carrying elements as defined in any of claims 5 to 22 wherein said Yersinia sequence elements are exchanged by a sequence element or signal specific for the strain used or compatible therewith.

60. A secretion type III carrying bacterial strain for the delivery of heterologous proteins into target cells according to claim 58 transformed with an expression vector according to claim 59.
